(19) United States
(12) Patent Application Publication
(10) Pub. No.: US 2006/0281134 A1
(43) Pub. Date: Dec. 14, 2006

(54) Method for analyzing biological data sets

(75) Inventor: Bradley Steven Love, Timonium, MD (US)

Correspondence Address: BIOTECHNOLOGY LAW GROUP;C/O PORTFOLIOIP, PO BOX 52050, MINNEAPOLIS, MN, 55402, US

(73) Assignee: Invitrogen Corporation, Carlsbad, CA (US)

(21) Appl. No.: 11/446,562

(22) Filed: Jun. 1, 2006

Related U.S. Application Data
(60) Provisional application No. 60/686,574, filed on Jun. 1, 2005.

(51) Int. Cl. G01N 33/53; G06F 19/00

(52) U.S. Cl. 435/7.1; 702/19

(57) ABSTRACT

The present invention relates to methods for analyzing biological data sets, and more specifically for identifying biomolecular differences between biological samples. In particular, the present invention is based in part, on the discovery that Markov's Inequality, and in certain illustrative aspects Chebyshev's Inequality, can be used to analyze biological data sets to identify positive signals. The data sets are typically generated using a biological assay, such as a biomolecule array assay.

### CROSS-REFERENCE TO RELATED APPLICATIONS

The present application claims the benefit of U.S. Provisional Patent Application No. 60/686,574, filed Jun. 1, 2005, by Bradley Steven Love; the entire content of this priority application is incorporated herein by reference.

### FIELD OF THE INVENTION

The present invention relates to methods for analyzing biological data sets, and more specifically for identifying biomolecular differences between biological samples.

### INTRODUCTION

Protein microarrays are emerging as an important new technology allowing for the discovery of novel biomarkers. The currently available protein microarray platforms vary markedly in the nature of their content. Antibody capture arrays containing a limited set of defined antibodies have particular utility in assaying for the presence of known biomarkers, but are not appropriate tools for the discovery of novel biomarkers. Peptide arrays and arrays containing denatured proteins have potential for increased content relative to antibody arrays, but are unlikely to interact with the full spectrum of autoantibodies due to the lack of conformational epitopes that are only available in full length native proteins.

Invitrogen's ProtoArray™ Human Protein Microarrays contain as many as 3,000 human proteins in defined locations immobilized on nitrocellulose-coated glass slides. Probing ProtoArrays™ with serum allows immediate identification of reactive proteins through a mechanism that is both simple and highly sensitive. Arrayed proteins are expressed in insect cells, and are therefore expected to contain appropriate posttranslational modifications. The majority of proteins are expected to be functional and in their native conformations (12). Therefore, ProtoArrays™ provide an ideal methodology for the identification of new autoantigen disease biomarkers.

Complex biological mixtures including serum can be analyzed for the presence of differentially expressed proteins in disease vs normal individuals. However, it is an art-recognized problem that current approaches such as mass spectrometry are biased towards high abundance proteins, and often fail to identify well established biomarkers such as PSA in certain assays (17).

As a solution to this problem, the present invention provides a method for reliably distinguishing positive signals on a protein array from false positives and negative controls using a stringent statistical method for analyzing the data.

### SUMMARY OF THE INVENTION

The present invention is based in part, on the discovery that Markov's Inequality, and in certain illustrative aspects Chebyshev's Inequality, can be used to analyze biological data sets to identify positive signals. The data sets are typically generated using a biological assay, such as a biomolecule array assay, which in illustrative embodiments provided herein, is a protein array assay. However, it will be understood that the methods provided herein, although exemplified for analyzing protein array data sets, can be applied to other biomolecule data sets as well.

A precision value calculated using Markov's Inequality in certain preferred embodiments of methods provided herein, utilizes negative control information, most preferably negative control information generated at the time of performing a method. By using Markov's Inequality to analyze biological data sets no distributional assumptions are made about the negative control distribution other than that the data set being collected is from an independently and identically distributed random sample. The methods provided herein utilize a probabilistic method based, in certain embodiments, on negative control values, to identify a positive signal on the array.

More specifically, Markov's Inequality values can be used to identify the presence or absence of a molecule, typically a biomolecule such as a peptide, protein or nucleic acid, in a biological sample, and to detect differences in the presence or absence of a biomolecule between types of biological samples. For example, in one embodiment, the present invention provides a method for determining the presence in a biological sample of a binding partner with affinity for a protein and/or peptide immobilized on a protein and/or peptide array. In certain illustrative methods provided herein, the binding of the binding partner to the protein or peptide thus identifies the protein or peptide and/or the binding partner as a biomarker. In one embodiment, a plurality of proteins and/or peptides and a negative control, all of which are immobilized on a protein and/or peptide array, are contacted with a biological sample. The signal generated for each immobilized protein and/or peptide and the negative control, after being contacted with the biological sample is used to determine a probability value (p-value) for each immobilized protein and/or peptide using Markov's Inequality, preferably Chebyshev's Inequality, to produce a Chebyshev's Inequality Precision Value (CI-p-Value). The CI-p-Value is calculated for each immobilized protein and/or peptide on the array by comparing the signal generated after contacting the immobilized protein or peptide with the sample with each protein on the array and a signal generated for a negative control on the array, where a p-value below a threshold p-value identifies a significant positive interaction with a particular protein on the array.

In another embodiment, a method for comparing the expression of binding partners between biological sample types is provided. In one embodiment, a method is provided for determining whether a binding partner is present in a biological sample or type of biological samples in a different frequency than in another biological sample or type of biological samples. In carrying out the method, each sample is individually contacted with a plurality of proteins immobilized on a protein array and interaction between one or more binding partners in the biological sample and one or more immobilized proteins is detected. Each interaction on the array is then assiged a probability value using Markov's Inequality. For example, Chebyshev's Inequality can be used to produce a Chebyshev's Inequality Precision Value (CI-p-Value), as described above. Significant CI-p-Values are then determined using a dynamic significance calculation calculated by identifying a minimum observed CI-p-Value for each sample for each protein on each array. Finally, the CI-p-Values are compared between biological sample types to identify differences between the sample types.

Other embodiments of the present invention will be evident from the following description and claims.

### BRIEF DESCRIPTION OF THE DRAWINGS

FIG. 1. Protocol for Serum Profiling Application. Briefly, ProtoArrays™ are blocked with a nonspecific protein blocker, treated with serum and incubated for 2 hours. Afterwards, the slides are washed to remove any unbound proteins/antibodies. The bound sera antibodies are then detected with anti-isotype specific secondary antibodies conjugated to AlexaFluor 647.

FIG. 2. Sensitivity of Detection. A graph of signal minus background values for the probings of custom protein arrays containing a gradient (0.4, 1.56, 6.25, 25 and 100 picograms) of NY-ESO-1 protein with a dilution series of serum known to be NY-ESO-1 seropositive. Dilutions are 1-150, 1-1000, 1-5000, 1-10000 and 1-50000 in probe buffer (see Materials and Methods). Purified glutathione-S-transferase (GST) is the negative control (100 picograms). A slide was also probed with only the secondary detection reagent (anti-Human neg).

FIG. 3. NY-ESO-1 Reactivity of Blinded Serum samples. Custom protein arrays containing a gradient of NY-ESO-1 antigen probed with 5 sera samples, two characterized for seroreactivity, Mel_{—}1-seropositive and Mel_{—}2-seronegative and three characterized for seroreactivity but blinded during study; Mel_{—}3, Mel_{—}4 and Mel_{—}5. Images and quantitation of signals are shown for each serum sample.

FIG. 4. Experimental Reprodubility. ProtoArray™ Human Protein Microarrays were probed in duplicate with serum from a healthy patient and with serum from a melanoma patient. The signals (signal-background values) for all human proteins on each array were plotted. A. correlation plot for the normal treated slides. B. correlation plot for the melanoma treated arrays.

FIG. 5. Identification of Potential Protein Biomarkers. The data from arrays probed with serum from a healthy individulal (x-axis) and with serum from a patient with melanoma (y-axis) was plotted. Note the lack of correlation for the two slides (R2 =0.35)

### DETAILED DESCRIPTION

Provided herein is an important new tool for analyzing biological data sets that can be used for immune response profiling, biomarker discovery and analysis, detection or diagnosis of a disease condition, or measuring the effects of a course of treatment on the disease state, among other uses. The methods provided herein allow for the immediate identification of statistically significant data relating to the presence of a biomolecule such as a protein, peptide, nucleic acid or other material in a biological sample.

In certain embodiments, the present invention provides a reproducible statistical approach for analyzing protein, peptide, and/or nucleic acid array expression data, data related to the presence of binding partners, especially antibodies, in samples, and changes in the presence or absence of these bi8nding partners in different samples. In other embodiments, the present invention provides a method for identifying statistically significant biomarkers indicative of a disease state in a biological sample such as, for example, human serum. Related embodiments further provide for the identification of biomarkers indicative of a disease state such as an autoimmune condition, a microbiological infection, cancer, a neurological disorder, a circulatory disorder, or a respiratory disorder, among other disorders. The methods can also be applied to the analysis of disease treatment regimens, where changes in biomarker expression are analyzed following treatment with a particular drug, therapy or vaccine, for instance, with changes in biomarker expression indicating whether or not the particular treatment regimen is effective in a patient. Thus, the present method is applicable to both biomarker discovery per se as well as biomarker analysis with respect to the detection, diagnosis, prognosis, and treatment of disease. Other embodiments are provided herein.

In one embodiment, a plurality of proteins and/or peptides and a negative control(s), all of which are immobilized on a protein and/or peptide array, are contacted with a biological sample. The signal generated for each immobilized protein and/or peptide and the negative control, after being contacted with the biological sample is used to determine a probability value (p-value) for each immobilized protein and/or peptide using Markov's Inequality, preferably Chebyshev's Inequality, to produce a precision value, such as a Chebyshev's Inequality Precision Value (CI-p-Value). The CI-p-Value is calculated for each immobilized protein and/or peptide on the array by comparing the signal generated after contacting the immobilized protein or peptide with the sample and a signal generated for a negative control on the array, where a p-value below a threshold p-value identifies a significant positive interaction with a particular protein on the array. This method for analyzing biological data sets is useful for the identification of biomarkers and/or for immune response profiling.

As is known in the art, a biomarker is a biochemical characteristic that can be used to measure the existence, prognosis or progress of disease or the effects of treatment. Typically, such biomarkers are recognized by detecting a biomolecule such as a protein, peptide, nucleic acid or other material in a sample, preferably but not necessarily a biological sample, that binds to the biomarker. Potential biomarkers that can be detected by methods provided herein include peptides, proteins or nucleic acids, among other biomolecules, and in illustrative embodiments are protein and/or peptide biomarkers. It is useful to note the difference in data types that result from DNA microarray expression analysis and protein array expression analysis. DNA microarrays generate a range of values in which the signal intensity is thought to correspond directly to the number of transcripts. Protein microarrays generate data that typically must be evaluated for the presence or absence of a significant signal. These two data types, known as continuous numerical data and dichotomous indicator data respectively, necessitate fundamentally different statistical approaches. The methods provided herein are particularly well-suited for dichotomous indicator data, such as protein or peptide array data With respect to protein biomarkers, the proteins may be cell membrane proteins, cytoplasmic proteins, secreted proteins, nuclear proteins, and the like, or may be binding partners such as antibodies. Many types of nucleic acid biomarkers, including DNA, RNA and variations thereof (i.e., protein-nucleic acids or PNAs) can be identified in practicing this invention. For instance, in certain embodiments a cDNA, mRNA, fragment thereof, or oligonucleotide corresponding thereto can be probed with a sample to identify nucleic acid biomarkers. It is further understood by those of skill in the art that many suitable biomarkers are well-known and widely available. As described below, potential biomarkers are typically affixed to a solid support prior to exposure to the sample.

In one embodiment, provided herein is a method for immune response profiling. This method identifies proteins in an on-test set of proteins, such as proteins on a microarray (e.g., human or yeast protein features) that are bound by an exogenous antibody added as an unique reagent or as part of the complex mixture of antibodies contained in serum. The methods provided herein can be used to analyze results from experiments where any bound, exogenous antibody is detected by probing with a second, Igclass specific antibody labeled with a fluorescent probe such as Alexa Fluor®647. Binding of the primary antibody on the microarray is then quantified by measuring the fluorescence intensity of each feature on the slide.

The basic approach employed by the method to analyze for binding of one or more biomolecules of a sample with one or more proteins of a set of proteins, such as proteins immobilized on a protein array, is as follows:

1. Calculate the appropriate fluorescent signal values taking into account corrections for background and the negative control features (i.e. spots) on the microarray;

2. Calculate CI P-Values of all the corrected intensities of the protein or peptide features (i.e. spots)

3. Identify the features that have a CI P-Value less than a user-defined cut-off value (e.g., one divided by the number of protein features on the microarray by default). These are the protein features that the methods provided herein score as positive for binding a binding partner in the sample, such as an antibody in the sample.

Step 1 above, calculation of appropriate fluorescent signal, is performed typically using commercially available software such as GenePix™ and/or Prospector™ (Invitrogen Corp., Carlsbad, Calif.). As will be understood, during this step outlier values for control replicates can optionally be removed, and background fluorescence can be optionally subtracted from signal values.

In another embodiment, a method is provided, for determining whether a binding partner is present more frequently in a first biological sample type or a second biological sample type from a plurality of biological samples of each biological sample type. The method includes contacting each sample individually with a plurality of proteins or peptides immobilized on a protein array, then calculating a probability value for each sample for each protein or peptide on each array using Markov's Inequality based on a comparison of a signal generated from the interaction of biomolecules in each sample with each protein on each array and a signal generated for a negative control on each array. In certain preferred embodiments, a dynamic significance calculation is used to identify proteins or peptides immobilized on the array that yield significant probability values, by identifying a minimum observed probability value for each sample for each protein or peptide on each array. For each protein or peptide, it is determined whether a significant probability value is observed more frequently in samples of the first biological sample type or samples of the second biological sample type. The Markov's Inequality value in certain illustrative example, is Chebyshev's Inequality precision value (CI-p-Value).

In one embodiment, a biomarker may be identified using a sample, preferably a biological sample such as human serum, by detecting binding partners, typically biomolecules (e.g., autoantibodies), present in the sample that bind to and/or react with the biomarker (e.g., an autoantigen). In certain disease states including autoimmune diseases and cancer, autoantibodies are expressed at altered levels relative to those observed in normal healthy individuals. For example, MAGEIA and NY-ESO-1 are well-established biomarkers and antibodies reactive to these proteins are observed in many melanoma patients. Biomarkers need not be expressed in a majority of disease individuals to have clinical value. For example, the receptor tyrosine kinase Her2 is known to be over-expressed in only 25% of all breast cancers, and yet is a clinically important biomarker of disease progression as well as specific therapeutic options. In one embodiment, the present invention provides a method for identifying biomarkers with statistically significant correlation with certain disease states.

In most embodiments of the present invention, a protein or nucleic acid of interest (i.e., a potential biomarker) is associated with a solid support prior to analysis, for example as a microarray. It is preferred that the solid support comprise glass, ceramics, nitrocellulose, amorphous silicon carbide, castable oxides, polyimides, polymethylmethacrylates, polystyrenes, gold or silicone elastomers. In one embodiment, the surface of the solid support is a flat surface, such as, but not limited to, glass slides. High density protein arrays can be produced on, for example, glass slides, such that chemical reactions and assays can be conducted, thus allowing large-scale parallel analysis of the presence, amount, and/or functionality of proteins. In a specific embodiment, the flat surface array has proteins bound to its surface via a 3-glycidooxypropyltrimethoxysilane (GPTS) linker.

In certain embodiments, it is preferred that the potential biomarkers are organized on the solid support, which in some embodiments is a high density array, for example protein arrays that include at least 100 proteins/cm² and preferably contain at least 500 protein/cm². In preferred embodiments, the potential biomarkers are organized as a positionally addressable array (i.e., a “chip”). In some embodiments, the array may include a plurality of immobilized proteins such as those found on the human or yeast-ProtoArray products available from Invitrogen Corp. (Carlsbad, Calif.). For example, the protein array can include 100, 250, 500, 1000, 2000, 2500, 5000, 7500, or 10,000 proteins from an organism, or all expressed proteins from an organism. In one embodiment, the present invention provides a positionally addressable array comprising a plurality of proteins, with each protein being immobilized at a different position on a solid support, wherein the plurality of proteins comprises at least 1, 2, 3, 4, 5, 10, 20, 30, 40, 50, 100, 200, 500, 1000, 1500, 2000, 2500, 3000, 4000, 5000, 6000, 7000, 8000, 9000, 10,000, 100,000, 500,000 or 1,000,000 protein(s). It is preferred that the origin on the proteins to be studied is mammalian, and more preferred that the mammal is a human being. Suitable high density protein arrays are available to one of skill in the art, such as Invitrogen's ProtoArrays™ (Invitrogen Corporation, Carlsbad, Calif.), among others.

In most cases, the methods described herein include a “negative control”. In certain embodiments, the negative control refers to a biomolecule unrelated to the potential biomarker of interest. Negative controls are typically molecules, such as proteins, that are known or believed not to interact with a molecule in an on-test sample. In methods provided herein, both the negative control and the potential biomarker are exposed to the same biological sample(s). Where a microarray is utilized, the array containing potential biomarker spots includes negative control spots containing protein, peptide(s), or nucleic acids unrelated to the potential biomarker protein, peptide, or nucleic acid (e.g. DNA). Typically, but not necessarily, the negative control and the potential biomarker are affixed to the same array. In either case, the same biological samples are contacted with both the negative control spot and the test spot to generate signal values for analysis using the methods described herein.

In other embodiments, the term negative control sample may refer to a type of biological sample (e.g., normal serum). Such a biological sample may also be referred to as the control or baseline biological sample. Suitable control or baseline biological samples include samples obtained from a person without the disease of interest or a person not treated with the treatment regimen of interest, for example. An exemplary suitable control/baseline biological sample would be serum obtained from a person without the disease of interest (e.g., the person does not have cancer). Data generated using test biological sample is then compared to that obtained for control/baseline sample in performing the analyses described herein. Where an array is utilized, identical proteins (i.e., potential biomarkers) are spotted onto one or multiple arrays and the arrays are then separately exposed to both control/baseline and test biological samples. The interaction data (i.e., signal value) is then compared between the two samples, with the negative control being the array exposed to the control/baseline sample (i.e., serum from the person without the disease of interest).

In certain other embodiments, both negative controls and negative control samples can be included in a particular experiment. This allows one to control for both non-specific interactions and differences in the binding of various biological samples. Furthermore, the negative controls for methods using arrays includes spots of negative controls, each of which can include different immobilized molecules, such as an unrelated protein or a buffer, and the signal values from these different immobilized molecules can be combined. The negative control in certain embodiments can be a molecule, such as a biomolecule, for example a peptide or protein that is present on some, many, most, and preferably all, potential biomakers (e.g., fusion proteins) immobilized on an array. This molecule can also be used to purify and/or detect the peptide or protein.

Whether the negative control is a biomolecule unrelated to the potential biomarker of interest or a control biological sample, a suitable number of controls must be included in the assay. For example, between 2 and 10,000, or between 4 and 1000 negative controls can be utilized. Where an array is utilized, one or more negative control is disposed on the array in a plurality of spots, for example, at least 2, 3, 4, 5, 6, 7, 8, 9, 10, 15, 20, 25, 50, 100, 250, 300, 400, 500, 750, 800, 1000, 1152, 1250, 1500, 1536, 1750, 2000, 3000, 4000, 5000, 7500, or 10,000 spots. For example, between 2 and 5000 , between 4 and 2500, between 6 and 2000, between 100 and 2000, between 250 and 2000, between 500 and 2000, between 1000 and 2000, between 1000 and 25000, between 1000 and 3000, between 1000 and 4000, between 1000 and 5000, or between 8 and 1000, total control spots can be utilized. Values of signals generated from the plurality of negative control spots are used in the equations provided herein. In certain illustrative examples, the negative controls include between 50 and 1000, for example 96 or 480 spots of buffer, which can be the same buffer as that which contains controls proteins to be immobilized; Between 200 and 500 spots, for example 288 spots of BSA; and/or between 250 and 1000 spots of GST, for example 768 spots.

By analyzing a plurality of negative control spots the Markov Inequality equations provided herein provide a better estimate of the population of negative spots such that a more accurate result can be obtained to determine whether an on-test spot is not a member of the negative spot population at a certain confidence level.

Illustrative methods of the present invention are based, in part, on the following three negative control-related principles
1. Negative Control information is preferably collected at the time of the experiment;
2. No distributional assumptions are made about the negative control distribution, other than the data that is being collected is a random sample that is independently and identically distributed; and
3. A probabilistic method based on the negative control distribution, will determine a positive biomarker, based on a predefined cut-off value.

The negative controls are determined by the particular experiment being performed. When using a protein array, such as the Invitrogen ProtoArray™ platform (Carlsbad, Calif.), the negative controls can include, for example, buffer, BSA and GST negative control spots. The CIP Value is the maximum probability that a protein/antibody/antigen signal comes from the negative distribution. There are various methods that can be utilized to use the CIP values for the detection of potential biomarkers of various levels of prevalence. 2-400, 4-20, 8-12

Methods provided herein, for identifying a positive signal, although illustrated with protein interaction data using a protein array, can be applied to virtually any type of biological assay, especially high throughput assays. For example, the methods can be used in conjunction with protein or nucleic acid gel-based assays as well as enzymatic or other detectable assays performed in microwell plates. In preferred examples, the biological assay includes the use of negative controls to generate CI-p Values. For example, the methods can be used to analyze data sets from signals generated by contacting a protein microarray with an enzyme. In this type of enzymatic assay, negative controls can include, for example, proteins that are known not to be substrates for the enzyme.

The present invention is based in part upon the use of statistical calculations. To perform such calculations, the following concepts are applicable:
Hypothesis Testing: This phrase means that two mutually exclusive hypotheses are given, one is typically called the null hypothesis and the other is typically called the alternative hypothesis. Data is then collected to test the viability of the null hypothesis, and this data is used to determine if the null hypothesis is rejected or not.
Rejection Rule: This is a statistical method in which the observed data either rejects the null hypothesis or fails to reject the null hypothesis. It is important to note that this Rule will never “accept the null or alternative hypothesis”; it is exclusively a rule to reject.

There are four possible outcomes to this approach, based on the true nature of the null hypothesis, and what is decided by the Rejection Rule. The four outcomes are shown in the following Outcomes Table.

Outcomes Table.

| | True Nature of H₀ | | |
|---|---|---|---|
| | | H₀ is True | H₀ is False |
| Decision by the | Reject H₀ | Type I Error | Correct Decision |
| Rejection | Fail to Reject H₀ | Correct Decision | Type II Error |
| Rule | | | |

Note that the true nature of Ho is never really known. The actual formula for the Rejection Rule varies from hypothesis test to hypothesis test depending on the type of data, and the set of assumptions being made.
Type I Error: Typically, the probability of a Type I error is denoted as α. In general this is considered the most serious type of error to make.
Type II Error: Typically the probability of a Type II error is denoted as β. Though this is also an error, it is usually controlled by attempting to minimize the probability of Type I Error.
Precision: Precision is the probability of not making a Type I Error. This can be considered as the probability of a true positive. Hence this is denoted as 1−α.
Power: Power is the probability of not making a Type II Error. This can be considered the probability of a true negative. Hence this is denoted as 1−β.

A statistical value calculated in certain embodiments of the present invention is the probability value or p-value. The p-value indicates the probability that the result obtained in a statistical test is due to chance rather than a true relationship between measures. Small p-values indicate that it is very unlikely that the results were due to chance. Therefore, if the p-value is small, statisticians would be confident that the result obtained is “real.” When p is less than 0.05 (P<0.05)—meaning that there is a less than 5% chance that the relationship is due to chance—statisticians usually conclude that the relationship is strong enough that it is probably not just due to chance. In certain embodiments, the invention is based on the calculation of Chebyshev's Inequality precision value, which is an application of Markov's Inequality as shown below.

In one embodiment, the present invention provides a method for identifying statistically significant interactions between a biomolecule such as a binding partner in a biological sample and a known potential biomarker such as a peptide, protein, or nucleic acid. As such, methods for identifying biomarkers correlating with a particular disease or other states for detection, diagnosis and or treatment of the disease are provided. Also provided are biomarkers identified using this method where the presence in a biological sample of a binding partner for the biomarker indicates the presence of a disease condition in the host from which the biological sample was obtained. A panel of two or more biomarkers is also provided, wherein the detection of multiple binding pairs of binding partner in the biological sample and biomarker in the panel (i.e. detection of an interaction between multiple binding partner in the biological sample and multiple biomarkers in the panel) indicates the presence of, or is diagnostic or prognostic for a disease condition in the host from which the biological sample was obtained.

The data analysis methods provided herein allows one to reliably identify specific proteins that are more frequently expressed in either disease or healthy populations. In order to do this, in illustrative embodiments, results are analyzed in a 3-step analysis process: 1) signals from each protein on each array is assigned a p-value, with the primary data output being a CI-p-Value (defined below); 2) each signal is then analyzed to determine whether or not it is statistically significant; and, 3) two biological samples are then compared to determine differences between significant protein binding signals between the two samples to distinguish, for example, patients having a particular diseas from healthy patients.

Step 1 in the analysis process is performed following contacting a biological sample with one or more potential biomarkers such as a peptide, proteins or nucleic acid. In illustrative aspects, the biological sample is brought into contact with a plurality of potential biomarkers such as peptides, proteins or nucleic acids immobilized (i.e. deposited) in an addressable and known location on a solid support (i.e., on an array). Incubation (i.e. exposure) is performed under conditions amenable to the binding of such binding partners to such pre-arranged proteins, as illustrated in the Examples provided herein.

When using a solid support such as an array, the interaction of a biomolecule from the sample with a potential biomarker on the array generates a signal from what is known in the art as a “spot”. A signal can also be generated when an enzyme present in a biological sample contacted with the array, catalyzes a detectable modification of a protein on the array, such as phosphorylation of a substrate. Known methods for labeling molecules in samples contacted with one or more proteins or peptides, such as by labeling molecules in a sample with a fluorescent or radioactive tag, can be utilized. Furthermore, methods for detecting the labeled molecule are well known in the art.

In performing step 1 of the above illustrative analysis process, p-values based on the signal from the protein spots on the array compared to negative controls are calculated for each protein on each protein array. In preferred examples, the primary output of the data analysis method provided herein is the Chebyshev's Inequality Precision Value (CI-p-Value), a type of p-value based on ‘Chebyshev's Inequality’ ((P. Chebyshev, Journal de Mathematics Pures et Appliqu'ees 12, 177-184 (1867)). The CI-p-Value provides a conservative estimate of the probability that the signal observed for a protein spot is indistinguishable from negative control signals: the lower the CI-p-value, the greater the probability that the signal is not due to a random event. Chebyshev's Inequality is used in order to avoid making assumptions on the behavior of the negative control distribution, and provides an upper bound on the “true” probability of a type I error, from which we can calculate a lower (conservative) bound on precision.

In greater detail, the CI-p-Value may be determined as follows. The value is derived by testing the following hypothesis:
H₀: This spot comes from the Negative Control Distribution
Hₐ: This spot does not come from the Negative Control Distribution
To minimize assumptions about the negative control distribution, and hence the assumptions effects on the resulting p-values to test the given hypothesis, Chebyshev's Inequality is utilized and states that if X is a random variable where μ=E(X) is the mean, σ²=Var(X) is the variance where if k>1 then,

$$P ( \frac{\vert X - μ \vert}{σ} \geq k ) \leq \frac{1}{{k}^{2}}$$

This is an absolute bound on the probability under the null hypothesis. Thus, under the null hypothesis this is the most conservative p-value estimate. Again under the null hypothesis, it is assumed that the non-control spot comes from the negative control distribution where the sample mean and sample standard deviation are estimated from the signals from the negative controls. Using this Inequality, one calculates the CI-p-Value as,

$$CI - p - Value = \{ \begin{matrix}1 & {Y}_{k} \leq \overline{X} + s \\ {( \frac{s}{( {Y}_{k} - \overline{X} )} )}^{2} & {Y}_{k} > \overline{X} + s\end{matrix}$$

where the mean and the standard deviation are from the observed signals in the Negative Control distribution. Note that this is an upper bound on the true probability, since any assumptions of the distribution are not made.

Regarding step 2 of the above illustrative analysis process, in one embodiment, a single CI-p-Value significance threshold is set. However, this static threshold setting has several limitations. For example, it does not allow two populations to be considered separately, and therefore will fail to detect potential biomarkers which consistently exhibit moderate reactivity (e.g., immunoreactivity) in the disease population. Applying a static threshold will identify only those proteins that elicit high levels of reactivity (e.g., immunoreactivity) in at least one sample, ultimately resulting in a relatively high false negative rate.

Therefore, step 2 preferably utilizes a dynamic significance calculation in order to avoid problems associated with static thresholding. As such, an acceptable level of precision for the experiment wide hypothesis test is initially set. In one embodiment, p<1/n is utilized, where n represents the number of different proteins on the array, or in certain illustrative embodiments, the number of protein spots on the array. This choice minimizes false positives due to multiple sampling required to correct for multiple testing error. Then, the individual observed CI-p-Values across all experiments for a given sample group (disease or healthy) for a given protein may be examined to find the minimal observed CI-p-Value, where the resulting level of precision of the experimental hypothesis test is greater than the set acceptable level of precision. This process sets the individual CI-p-Value level of precision, which is likely different for each protein within an experiment. A more detailed mathematical description of the dynamic binomial calculation follows.

The collected data may be modeled as a binomial random variable, which is parameterized as the probability of a success (p), and the number of independent trials (n). If the random variable is X (the number of successes), where the probability of success is 0<p<1, out of n independent trials, then the probability of observing x success is given by,

$$P ( X = x ) = ( \begin{matrix}n \\ x\end{matrix} ) {{p}^{x} ( 1 - p )}^{n - x} \\ = \frac{n !}{x ! ( n - x ) !} {{p}^{x} ( 1 - p )}^{n - x}$$

Given a probability of calling a protein a positive hit, when in reality it is not a positive hit, then the probability of observing at least x false positive hits is given by,

$$\begin{matrix}α = P ( X \geq x ) \\ = {\sum }_{i = x}^{n} P ( X = i ) \\ = {\sum }_{i = x}^{n} ( \begin{matrix}n \\ i\end{matrix} ) {{p}^{i} ( 1 - p )}^{n - i}\end{matrix}$$

where 1−α is the probability of not making a false positive error. Typically, a confidence level is set before the experiment to determine an acceptable probability of making a correct positive hit.
For an experiment with 1896 samples (see below), for example, this threshold is set at 1 divided by the number of array proteins, allowing for one false positive per array. Thus, if C₁, C₂, . . . , Cₙ are n ordered from largest to smallest CI-p-values, then the Dynamic Binomial attempts to find the value m such that

$${min}_{m} ( {\sum }_{i = 1}^{m} ( \begin{matrix}n \\ i\end{matrix} ) {{C}_{m}^{i} ( 1 - {C}_{m} )}^{n - i} < \frac{1}{1896} )$$

If the result is an m value where this is satisfied, then there are m significant hits where Cₘ is the chip CI-p-value cutoff value, where the “Disease” p-value is given by

$${\sum }_{i = 1}^{m} ( \begin{matrix}n \\ i\end{matrix} ) {{C}_{m}^{i} ( 1 - {C}_{m} )}^{n - i}$$

The analysis of ProtoArray™ data in this type of experiment derives its statistical power from examining the distribution of signals for any given protein in order to establish threshold p values that are specific to each population under study. As such, signals from individual samples cannot be compared between populations.

Following the determination of signal significance in step 2 shown above, proteins expressed at significantly different frequencies in a first biological sample type versus a second biological sample type (e.g., disease vs healthy populations) are identified in step 3. Signals which are observed more frequently in either healthy or disease sera are potential biomarkers (e.g., autoantigens or tumor markers). In limited samples sets (e.g., 10 healthy, 10 disease), fairly large differences in biomarker prevalence are required to reliably distinguish healthy from disease. For instance, using the confidence Table 1 below, a biomarker that is identified in 8/10 samples will correspond to a distribution in the population of 48.22%-93.98% (with 95% confidence). One must observe autoantigen signal in <1/10 healthy sera in order to distinguish this from healthy sera, since the corresponding 2.28%-41.28% distribution (with 95% confidence) is the highest frequency which does statistically distinguished from) the disease distribution.

TABLE 1
Confidence Intervals for Biomarker Prevalence

| | 90% Confidence | |
|---|---|---|
| Number Samples | Interval | 95% Confidence |
| Demonstrating Significant | for Prevalence of | Interval for |
| Immunoreactivity (n = 10) | Marker | Prevalence of Marker |
| 0* | 0-18.89% | 0-23.84% |
| 1 | 3.33%-36.44% | 2.28%-41.28% |
| 2 | 7.88%-47.01% | 6.02%-51.78% |
| 3 | 13.51%-56.44% | 10.93%-60.97% |
| 4 | 19.96%-65.02% | 16.75%-68.21% |
| 5 | 27.12%-72.88% | 23.38%-76.62% |
| 6 | 34.98%-80.04% | 30.79%-83.25% |
| 7 | 43.56%-86.49% | 39.03%-89.07% |
| 8 | 52.99%-92.12% | 48.22%-93.98% |
| 9 | 63.56%-96.67% | 58.72%-97.72% |
| 10* | 81.11%-100% | 76.16%-100% |

Based on 95% confidence intervals, the frequencies shown in Table 2 can be distinguished, regardless of which population corresponds to disease or normal samples. Of course, increasing the number of replicates increases the sensitivity of the assay. Furthermore, although a 90% confidence interval and a 95% confidence interval are analyzed, a confidence interval between 75% and 99.99% can be set, for example 75%, 80%, 85%, 90%, 95%, 96%, 97%, 98%, 99%, or 99.99%. in utilizing peptide, protein or nucleic acid arrays, typically the threshold probability cutoff is between 0.1 and 100 false positive errors per array. In preferred embodiments, the probability cutoff is between 0.5 and 2 false positives per array.

TABLE 2
Potential frequency distributions for two populations that allow
biomarker prevalence to be distinguished given a sample
size of n = 10 for each population.

| | Population 1 | Population 2 |
|---|---|---|
| Number of Samples | 10 | 0 |
| giving Significant Signal | 10 | 1 |
| (n = 10) | 10 | 2 |
| | 10 | 3 |
| | 10 | 4 |
| | 9 | 0 |
| | 9 | 1 |
| | 9 | 2 |
| | 8 | 0 |
| | 8 | 1 |
| | 7 | 0 |

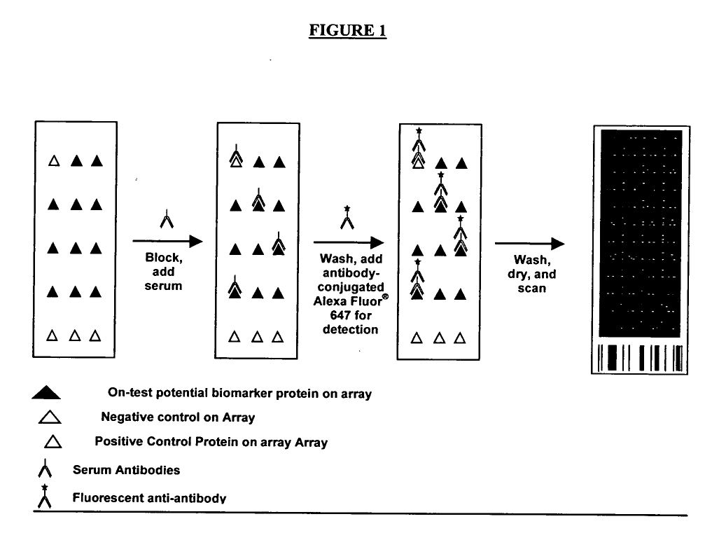

An exemplary method for serum profiling on high density protein arrays, such as ProtoArrays™ (Invitrogen Corporation, Carlsbad, Calif.) (FIG. 1) involves diluting serum in a profiling buffer and incubating dilute serum on the high density protein arrays. Autoantibodies present in the serum are detected by incubating arrays with an AlexaFluor™-conjugated anti-human IgG antibody. In this exemplary method, the protein arrays contain thousands of full length human proteins containing the majority of post-translational modifications, for example by being expressed in a eukaryotic cell such as an insect cell. Therefore, serum autoantibodies encounter potential biomarkers on the array in essentially the same conformations as those found in the body. Because the protein identity of each spot on the array is known, proteins that interact specifically with autoantibodies present in the serum of disease individuals with either elevated or reduced frequency relative to healthy individuals can be quickly identified. The amount of material required for serum profiling on ProtoArrays™ is ≦10 μ.

The methods provided herein are typically performed by one or more computer program or modules of computer programs. Accordingly, in certain embodiments, provided herein is a recordable computer medium that includes an executable computer program for performing a method provided herein. For example, a method for determining a positive interaction between a sample component, such as an autoantibody, and a biomolecule on an array with which the sample is contacted, such as a protein or peptide, as provided herein. The computer program can be executed while the program resides on a local computer, or while the programs reside on a server connected to a local computer, such as through a link on an Internet portal, as will be understood. The server can be a server that is connected to a computer as part of an intranet or an extranet. For example, in certain embodiments, the program resides on a server of a protein array provider that is accessed by a customer from the provider's Internet site.

In certain embodiments, the invention provides a computer program product for use in conjunction with a computer system, the computer program product comprising a computer readable storage medium and a computer program mechanism embedded therein, the computer program mechanism comprising instructions for performing a method provided herein. In certain embodiments, the instructions are computer-executable instructions in a computer language.

In certain embodiments, the invention provides a method implemented by a computer system coupled to a wide-area network, wherein the method is a method of the present invention. In certain, specific embodiments, the wide-area network is the internet. In other embodiments, the information can be stored on and retrieved from a disk.

The invention further provides a computer comprising a central processing unit; and a memory, coupled to the central processing unit, the memory storing: a) one or more data structures, said one or more data structures dimensioned and configured to provide instructions for performing a method provided herein.

In another embodiment, provided herein is a method for generating revenue by selling to a customer, a service for determining whether a binding partner is expressed preferentially in a first biological sample type or a second biological sample type from a plurality of biological samples of each biological sample type. The service can be performed at least in part, by a provider by performing a method that includes:

a) contacting each sample individually with a plurality of proteins or peptides immobilized on a protein array:

b) calculating a probability value for each sample for each protein or peptide on each array using Markov's Inequality based on a comparison of a signal generated from the interaction of biomolecules in each sample with each protein on each array and a signal generated for a negative control on each array;

c) identifying significant probability values using a dynamic significance calculation calculated by identifying a minimum observed probability value for each sample for each protein or peptide on each array; and

d) determining for each protein or peptide, whether a significant probability value is observed more frequently in samples of the first biological sample type or sample of the second biological sample type to identify binding partners expressed preferentially in one of the biological sample types.

In one illustrative example, a customer can contact a protein microarray with a biological sample and a labeled detection molecule, such as a labeled secondary antibody, or with a labeled probe, detect a signal generated by the label for each spot on the array, and upload to an Internet portal of a provider, an image of the protein microarray upon detection of a signal generated. An image analysis function and a data analysis function for performing methods provided herein can be provided to analyze the protein microarray image according to methods provided herein.

In illustrative methods for generating revenue by providing a service for performing the methods provided herein, for the samples profiled, array images, quantified spot data and processed results can be provided by a provider to a customer. Table 3 describes the format and contents of each result file in an illustrative service. An explanation of column headings of the cross-array results and summary spreadsheets is provided in Table 4.

TABLE 3
Result File Formats and Descriptions

| Results Files (format) | Result File Description |
|---|---|
| Biomarker Profiling | This document describing the results and experimental method of |
| Report (.pdf) | the Biomarker Profiling Experiment |
| IRBP Individual Sample | Processed profiling data for samples is provided, with data from |
| Results (.xls) | each sample or negative control given in individual worksheets. Z- |
| | factor, Z-score, NC P value (Negative Control P value), CI P value |
| | (Chebyshev's Inequality P value) and replicate CV for each of the |
| | human proteins printed on arrays used in this experiment. A “hit” |
| | “no hit” call is made based on a Z-score value≧3. Gene accession |
| | identifier, Invitrogen ultimate ORF Clone identifier, array position |
| | (block, row, column), gene identifier, protein concentration, and |
| | raw signal and background values are included. |
| IRBP Significance Results | Workbook of significant protein hits identified from array images |
| (.xls) | in this experiment based on the flexible binomial calculation of |
| | significance. The Flexible Binomial Summary table defines the |
| | number of proteins that were significant interactors in each study |
| | population. The Flexible Binomial Results worksheet contains the |
| | maximum P Value associated with each array protein for all |
| | populations in the stusy. Significant protein interactors observed in |
| | ≧1 disease sample and no normal samples are listed in individual |
| | worksheets. Summary includes gene accession identifier, |
| | Invitrogen ultimate ORF Clone identifier, number of samples |
| | exhibiting significant interaction for each population, maximal P |
| | value for each population, and gene description. |
| Protein Sequence File | Protein identifiers, description, protein sequence and protein |
| (.xls) | completeness (domain or full length) for the proteins printed on |
| | the arrays run in this experiment. |
| Quantified Array Spot | Text file produced from quantification of array spots in GenePix. |
| Values (.gpr in Gene Pix | |
| Results folder) | |
| Array List File (.gal in | File used in GenePix to associate spots on the array image with |
| Gene Pix Results folder) | feature identifiers. A separate array list file is produced from each |
| | array printing. All of the arrays used in this experiment were |
| | printed from the same print lot. |
| High Resolution Array | TIFF images for each array are provided. |
| Images (.tif in Array | |
| Images folder) | |

TABLE 4
Description of Column Headings in the IRBP Individual
Sample Results and IRBP Significance Results

| Column Heading | Description |
|---|---|
| Database ID | Database nucleotide accession number of the corresponding human |
| | protein on the array. |
| Ultimate ORF ID | Invitrogen's Ultimate ™ ORF Clone ID is provided for human array |
| | proteins derived from an Invitrogen Ultimate ORF Clone. Some |
| | kinase domains were cloned separately and have an empty field in |
| | this column. |
| Array ID | This number identifies the Block, Row, and Column for each protein |
| | on the array. |
| Signal | The pixel intensity calculated by GenePix for each protein on the |
| | array. |
| Background | The background pixel intensity calculated by GenePix for each |
| | protein on the array |
| Signal Used | The Signal minus Background value for each protein on the array. |
| | This number is used to calculate the Z-factor, the Z-score, and the NC |
| | P value and CI P value. |
| Z-factor | A confidence value that the Signal Used is significantly different than |
| | the negative control. |
| Z-score | The Signal Used value minus the mean Signal Used value from the |
| | negative control distribution, divided by the standard deviation of the |
| | negative control distribution. |
| NC P Value | The number of signals for the negative control distribution that are |
| | less than any given Signal Used, divided by the total number of |
| | negative control signals. |
| CI P Value | The probability that the Signal Used value comes from the |
| | negative control distribution. |
| Sample CV | Coefficient of variation for assay from duplicate spots. The standard |
| | deviation of the spot signals is divided by their mean spot intensity. |
| Sample Count | The number of samples that exhibit significant interaction with a |
| | given array protein. |
| Sample P Value | The maximal P value associated with a given array protein. |
| Concentration (nM) | Concentration of the human protein on the array determined by |
| | probing anti-GST antibody to known GST concentrations spotted on |
| | the array. The concentration of control proteins in ng/μl is appended |
| | to the name. Typically, these proteins lack GST domains. |
| Description | Database description for the nucleotide accession represented by the |
| | corresponding human protein on the array. |

A better understanding of the present invention and of its many advantages will be had from the following examples, given by way of illustration.

### EXAMPLES

### Example 1

### Materials and Methods

A. Purified proteins and Human Sera: Purified NY-ESO-1 protein, a monoclonal anti-NY-ESO-1 antibody (mouse) and human sera from healthy controls and patients with melanoma were generously provided by Dr. Ruth Halaban (Yale Univeristy).

B. Human Protein Collection: The majority of the human protein collection is derived from the human Ultimate™ ORF (open reading frame) Clone Collection available from Invitrogen (available on the Internet at orf invitrogen.com, incorporated by reference in its entirety). Each Ultimate™ ORF Clone is full insert sequenced and is guaranteed to match the corresponding GenBank amino acid sequence. Approximately 200 human proteins printed on the array represent the human protein kinase collection derived from full insert sequenced clones but are not Ultimate™ ORF Clones. (For accession number and amino acid sequence for each protein, download the Protein Information File from www.invitrogen.com/protoarray, incorporated by reference in its entirety).

All clones used to generate the human protein collection are entry clones consisting of a human ORF cloned into a Gateway® entry vector. Each entry clone was subjected to a LR reaction with the destination vector, pDEST™2 to generate an expression clone. The expression clone was then used to express the protein in insect cells as an N-terminal GST-fusion protein using the Bac-to-Bac® Baculovirus Expression System available from Invitrogen. After verifying that each clone expresses a protein of the expected molecular weight by western blotting, the proteins were expressed and purified using high-throughput procedures. The GST-tagged fusion proteins were purified under conditions optimized to obtain maximal protein integrity and function.

C. ProtoArraV™ Manufacturing: The purified human proteins were printed on nitrocellulose-coated slides in a dust-free, temperature and humidity controlled environment to maintain consistent quality of the microarrays. The arrays were printed using an automated process on an arrayer that is extensively calibrated and tested for printing ProtoArray Human Protein Microarrays. After production, each microarray was visually inspected for obvious defects that could interfere with the experimental results. To control for the quality of the printing process, several microarrays from each lot were probed with an anti-GST antibody. Since the proteins contain a GST fusion tag, probing the microarrays with an anti-GST antibody allows identification of irregular spot morphology or missing spots. The arrays were also functionally qualified by probing control proteins to detect the appropriate interactions.

D. Serum Profiling Assays on Protein Arrays: Slides were blocked with 1% BSA/PBST at 4° C. for 1 hour. Serum samples were diluted in probe buffer (1×PBS, 5 mM MgCl₂, 0.5 mM DTT, 5% glycerol, 0.05% Triton X-100, 1% BSA) and added to arrays under a Hybrislip™. The diluted samples were incubated at 4° C. for 120 minutes in a 50 ml conical tube and then transferred to an Evergreen Scientific Pap chamber. Arrays were washed three times; 8 minutes per wash with gentle shaking in 20 mls probe buffer. Subsequently, a solution of anti-human IgG conjugated to AlexaFluor647 (anti-Human IgG-Alexa Fluor™-647 (Invitrogen) 1.0 μg/ml) in probe buffer was added and incubated at 4° C. for 120 minutes. Arrays were washed three times (as above) and dried.

E. Data Acquisition/Analysis: Arrays were scanned with an Axon 4000B fluorescent scanner (Molecular Devices). Data was acquired with GenePix Pro software (Molecular Devices). Quantitated spot files were processed using Microsoft Excel and/or ProtoArray™ Prospector™ software (Invitrogen, Carlsbad, Calif.) to determine which proteins interact with biomolecules of the samples. The ProtoArray™ Prospector™ software incorporates background correction, CI-p-Value calculations using the formulas provided herein, and dynamic binomial calculations of significance.

### Example 2

### Experimental Results

ProtoArray™ Human Protein Microarrays are arrays of highly purified proteins immobilized on nitrocellulose coated slides. Proteins are expressed in Sf9 cells and are expected to contain many of the modifications observed in mammalian cells. Invitrogen ProtoArrays™ contain functionally immobilized proteins, present structured epitopes, (data not shown) and have been used for a variety of applications.

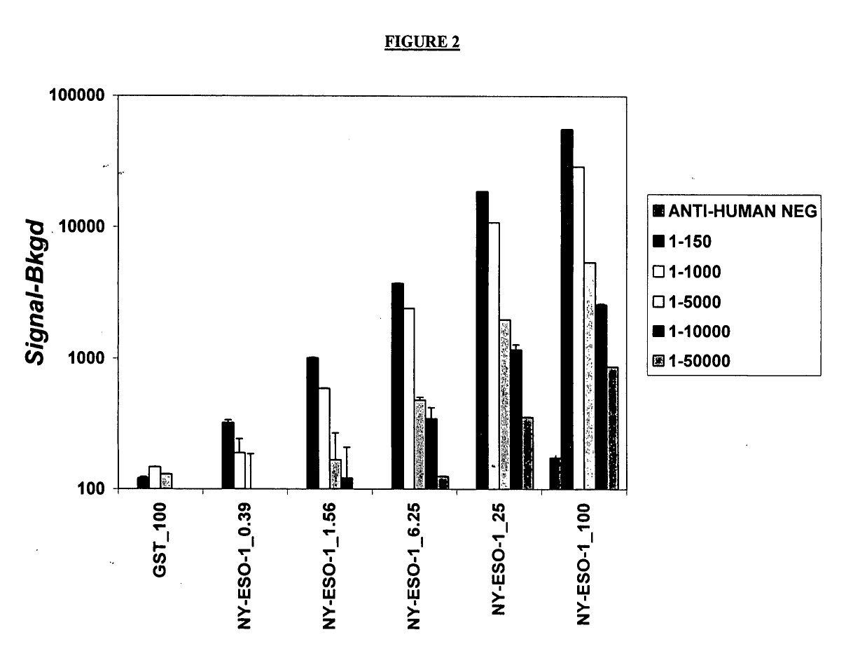

A. Use of Protein Arrays to Identify Biomarkers. FIG. 1 illustrates the basic protocol for using ProtoArray™ Human Protein Microarrays to identify elevated or decreased levels of antibodies in serum. Briefly, the protein arrays are blocked with a nonspecific protein blocker, incubated with diluted serum, washed, and then incubated with a secondary detection reagent to detect bound serum antibodies. In the experiments described below, we used an antibody that is conjugated to a fluorescent dye (Alexa Fluor™-647) that interacts with the Fc portion of human IgG antibody. To demonstrate the utility of protein microarrays for serum profiling to identify potential protein biomarkers, we probed custom protein arrays containing a gradient (approximately 0.4-100 picograms) of NY-ESO-1 protein with a serum sample known to be seroreactive against NY-ESO-1 protein. A 1:150 dilution of the serum sample produced a significant signal with as little as 0.4 picograms of NY-ESO-1 protein (FIG. 2). Significant signals were not observed with a negative control (purified glutathiose-S-transferase (GST), 100 picograms protein). Probing of parallel arrays with intermediate dilutions (1:150, 1:1,000, 1:5,000, 1:10,000 and 1:50,000) of the serum sample resulted in decreasing signals with decreasing amount of serum. Significant signal was observed on arrays containing as little as 25 picograms of recombinant NY-ESO-1 protein with serum dilutions up to 1:50,000. These results demonstrate that the serum profiling application protocol for detection of human antibody-protein complexes on protein arrays is both specific and highly sensitive.

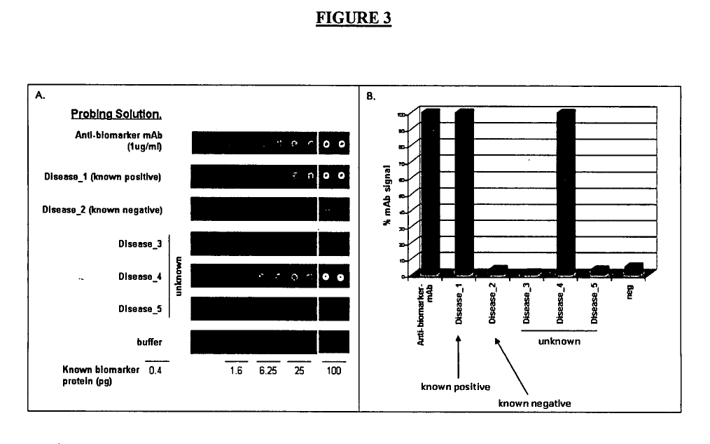

B. Protein Array Results Consistent with ELISA. NY-ESO-1 custom protein arrays were also probed with a panel of five serum samples derived from melanoma patients. The seroreactivity of all samples against the NY-ESO-1 protein had been previously established by ELISA at an independent laboratory. Sera included one sample identified by ELISA as NY-ESO-1 seropositive (Mel_{—}1), and another identified by ELISA as NY-ESO-1 seronegative (Mel_{—}2). The remaining three serum samples were blinded (Mel_{—}3-5). As shown in FIG. 3, the known seronegative and seropositive samples gave the expected signals on the arrays. Probings of the three blinded samples against the custom arrays demonstrated that sample Mel_{—}4 is seropositive and samples Mel_{—}3 and Mel_{—}5 were both seronegative. Unblinding of the results revealed that the seroreactivity for NY-ESO-1 on the arrays for these samples was consistent with the ELISA data.

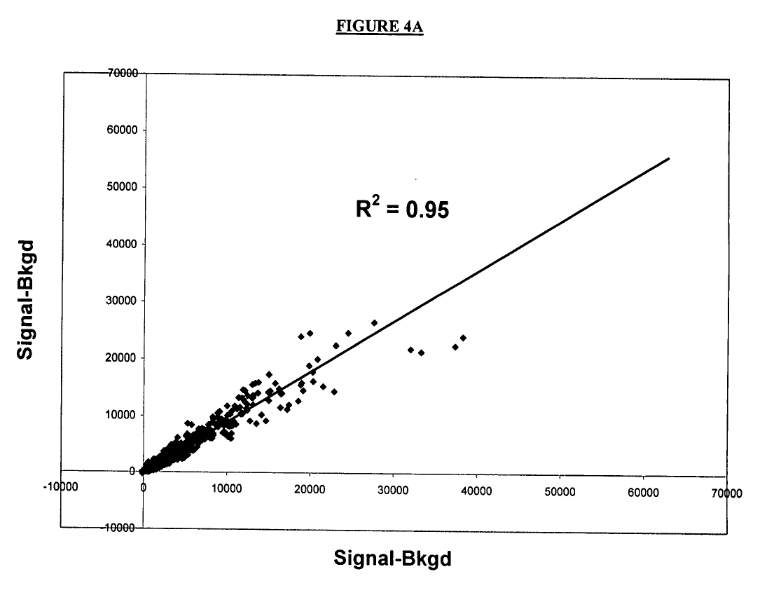

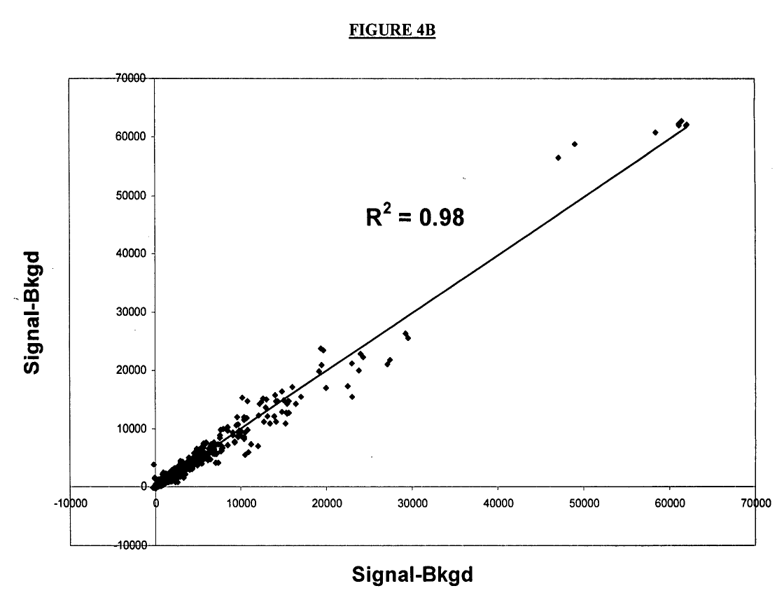

C. Performance Analysis of Serum Profiling Using Protein Arrays. To address the performance of the serum profiling application for identifying potential biomarkers, Human ProtoArray™ Protein Microarrays 1.0 nc containing about 2000 different human proteins were assayed in duplicate with serum samples from a normal/healthy patient and a patient with melanoma (Mel_{—}2). A plot of the signals from the replicate probings is shown in FIG. 4, demonstrating that the assay is highly reproducible (R² value of 0.95 for normals, FIG. 4A; R² value of 0.98 for melanoma serum, FIG. 4B; coefficient of variation for all human proteins on arrays<15%).

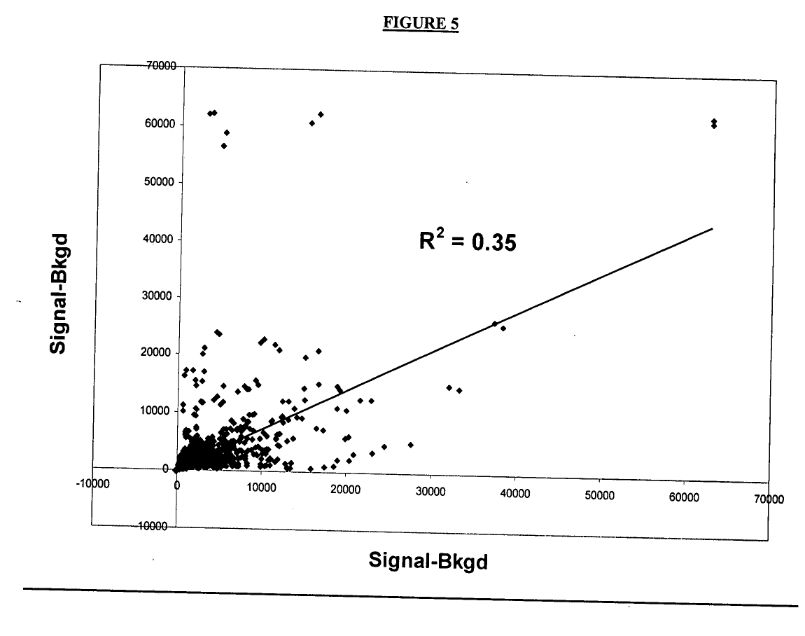

To identify potential biomarkers from these experiments, data from arrays probed with disease sera must be compared to data from arrays probed with sera from healthy individuals. In FIG. 5, a plot of the signals from an array probed with serum from a patient with melanoma versus an array probed with serum from a healthy patient exhibited little to no correlation (R² value of 0.23). The observed differences in antibody reactivity between these two samples could be due to disease-specific factors or other parameters such as age, sex, diet and environmental stimuli. Therefore, to confidently identify potential disease-specific biomarkers from the profiling of sera on Protoarray™ Protein Microarrays, the number and types of samples must be carefully considered and appropriate statistical algorithms must be employed. We observed from a larger study (melanoma serum samples, n=10) that several proteins known to be autoantigens for melanoma were in fact observed using this technology. Elevated signals for the proteins, MAGE4A, Rab38, SLC22A9, and SLC23A2 were observed in patients with melanoma, which is consistent with data obtained by ELISA (data not shown). Perhaps even more significant, a number of novel proteins (>50) were identified to have increased seroreactivity in patients with melanoma relative to healthy control samples.

### Example 3

### Chebyshev's and Markov's Inequality P-Values

The following Example discusses the Chebyshev's Inequality p-values (CIP) and Markov's Inequality p-values (MIP), their probabilistic background and their application in methods for analyzing samples for the presence of a biomolecule.

CIP Value

Assume that X₁, X₂, . . . , Xₙ, where there are n observed signals are from the negative control distribution, we can then calculate the following:
Sample mean:

$$\hat{μ} = \frac{1}{n} {\sum }_{i = 1}^{n} {X}_{i}$$

Sample standard deviation:

$$\hat{σ} = \sqrt{\frac{1}{n - 1} {\sum }_{i = 1}^{n} {( {X}_{i} - \hat{μ} )}^{2}}$$

The following Theorem is utilized:

Theorem 1—Assume that X₁, X₂, . . . , Xₙ are random variables that are independently and identically distributed with probability distribution function f(X), g is a function defined on [symbol] ⁿ→ [symbol] where g>0, also assume that k>0, then

$$P ( g ( {X}_{1} , {X}_{2} , K , {X}_{n} ) \geq k ) \leq \frac{E ( g ( {X}_{1} , {X}_{2} , K , {X}_{n} ) )}{k}$$

Proof Let A={X′∈ [symbol] ⁿ|g(X′)≧k} and let X=(X₁, X₂, K, Xₙ) then,

$$\begin{matrix}E ( g ( {X}_{1} , {X}_{2} , K , {X}_{n} ) ) = {\int }_{{ℜ}^{′′}} g ( X ) f ( X ) ⅆ {x}^{n} \\ {\int }_{A} g ( X ) f ( X ) ⅆ {x}^{n} + {\int }_{{A}^{c}} g ( X ) f ( X ) ⅆ {x}^{n} \geq \\ {\int }_{A} g ( X ) f ( X ) ⅆ {x}^{n} \geq k {\int }_{A} f ( X ) ⅆ {x}^{n} \\ = kP ( X ∈ A ) \\ = kP ( g ( {X}_{1} , {X}_{2} , K , {X}_{n} ) \geq k )\end{matrix}$$

Rearranging the above terms we get the result,

$$P ( g ( {X}_{1} , {X}_{2} , K , {X}_{n} ) \geq k ) \leq \frac{E ( g ( {X}_{1} . {X}_{2} , K , {X}_{n} ) )}{k} φ$$

With Theorem 1 in hand, Therum 2 may be utilized:

Theorem 2 (Markov's Inequality)—Assume that X is a random variable, where μ=E(X), then

$$P ( {\vert X - μ \vert}^{P} \geq {k}^{P} ) \leq \frac{E ( {\vert X - μ \vert}^{P} )}{{k}^{P}}$$

Proof—This is a direct application of Theorem 1 since |X−μ|^{r}≧0φ

Finally, Chebyshev's Inequality is shown,

Theorem 3 (Chebyshev's Inequality)—Assume that X is a random variable where μ=E(X), then

$$P ( \frac{\vert X - μ \vert}{σ} \geq k ) \leq \frac{1}{{k}^{2}}$$

Proof—The result is shown utilizing Markov's Inequality:

$$\begin{matrix}P ( \frac{\vert X - μ \vert}{σ} \geq k ) = P ( \vert X - μ \vert \geq k σ ) \\ = P ( {\vert X - μ \vert}^{2} \geq {k}^{2} {σ}^{2} ) \\ \leq \frac{E ( {\vert X - μ \vert}^{2} )}{{k}^{2} {σ}^{2}} \\ = \frac{{σ}^{2}}{{k}^{2} {σ}^{2}} \\ = \frac{1}{{k}^{2}}\end{matrix}$$

Chebyshev's Inequality may then be used to build an algorithm for detecting protein signals:

Theorem 4—Assume that k−μ≧σ, where μ and σ is the mean and standard deviation respectively of the observed data then,

$$P ( X \geq k ) \leq \frac{{σ}^{2}}{{( k - μ )}^{2}}$$

Proof—Assume that μ=E(X) and σ²=Var(X), where X is a random variable, then by applying Chebyshev's Inequality we have,

$$\begin{matrix}P ( X \geq k ) = P ( X - μ \geq k - μ ) \\ = P ( \vert X - μ \vert \geq \vert k - μ \vert ) \\ = P ( \frac{\vert X - μ \vert}{σ} \geq \frac{\vert k - μ \vert}{σ} ) \\ \leq \frac{1}{{( \frac{\vert k - μ \vert}{σ} )}^{2}} \\ = \frac{{σ}^{2}}{{( k - μ )}^{2}}\end{matrix}$$

It is useful to note that Theorem 4 gives an upper bound of the actual probability that a random variable is greater then some value k. This interpretation of Theorem 4 is utilized, and the signals from the negative control distribution and define the CIP Value.

Definition—Any signal denoted as X from any non-control spot is defined as,

$$CIP Value = \{ \begin{matrix}\frac{{\hat{σ}}^{2}}{{( X - μ )}^{2}} & X - \hat{μ} \geq \hat{σ} \\ 1 & otherwise .\end{matrix}$$

The following hypothesis test is then designed:
H₀: The marker is not significantly expressing in a state
Hₐ: The marker is significantly expressing in a state
   A significant cut off level is denoted as a, and is an acceptable probabilistic cut-off value to determine the detection of a present biomarker.

Using the rejection rule based on Theorem 4 and the assumptions under the null hypothesis, the above-defined CIP Value is utilized. Specifically, information is collected on the negative control values. Within the exemplary protein array platform, this includes collecting the signals from spots on the protein arrays that contain only buffer, BSA or GST. From the negative control distribution, the sample mean \hat{μ} and sample standard deviation \hat{σ} are determined, and the CIP-Value is calculated as defined above.

Rejection Rule—The rejection rule for the above hypothesis test is given as, reject H₀ if

$$\frac{{\hat{σ}}^{2}}{{( X - μ )}^{2}} \leq α$$

and fail to reject H₀ otherwise if

$$\frac{{\hat{σ}}^{2}}{{( X - μ )}^{2}} > α$$

fail to reject H₀ where X is the observed signal for the protein. Since α is user set, this will allow the user to control for multiple testing issues that arise for high throughput array methods. Additionally since the CIP-Value is a probability, these values can be modified for multiple testing as well.

### Example 4

Identification of Potential Biomarkers on Protoarrays™ Using Methods Provided Herein.

The following Example illustrates that the methods provided herein can be used to effectively identify biomarkers. Twenty serum samples provided by Yale University and the Ludwig Cancer Institute at Sloan Kettering, were profiled, including 10 samples from healthy individuals and 10 samples obtained from individuals diagnosed with melanoma. All serum samples demonstrated good signal to background on arrays. Median signals for human proteins and negative control proteins used for signal normalization (refer to Experimental Methods) were similar to the negative control assay, indicating that the samples did not interact non-specifically with components of the system.

Experimental Methods

Human Protein Collection

Human clones were obtained from Invitrogen's Ultimate™ ORF (open reading frame) collection or from a Gateway® collection of kinase clones. The nucleotide sequence of each clone was verified by full length sequencing. All clones were transferred into a system for expressing recombinant proteins in insect cells via baculovirus infection. Using a high-throughput insect cell expression system, thousands of recombinant human proteins were produced in parallel. Each protein is tagged with Glutathione-S-Transferase (GST), which enables high-throughput affinity purification under conditions that retain activity. All steps in the process are carried out at 4° C. which in combination with the overall speed of the high-throughput purification process helps to ensure that proteins are purified in a functional form. After purification, a sample of every purified protein is checked by Western blot to ensure that the majority of protein is present at the predicted molecular weight. In addition, all proteins are printed onto arrays and the concentration of each protein is determined.

ProtoArray™ Human Protein Microarray Manufacture

The output of the protein purification process described above is thousands of purified proteins that are ready to be printed on arrays. A contact-type printer equipped with 48 matched quill-type pins is used to deposit each of these proteins along with a set of control proteins in duplicate spots on 1″×3″ glass slides that have been derivatized with chemicals to promote protein binding. The printing of these arrays is carried out in a cold room under dust-free conditions in order to preserve the integrity of both samples and printed microarrays. Before releasing protein microarrays for use, each lot of slides is subjected to a rigorous quality control (QC) procedure, including a gross visual inspection of all the printed slides to check for scratches, fibers and smearing. Since each of the proteins is tagged with GST, a GST-directed antibody detects human proteins in a second QC assay. The procedure measures the variability in spot morphology, the number of missing spots, the presence of control spots, and the amount of protein deposited in each spot. The arrays are designed to accommodate 12,288 spots. Samples are printed in 200 mm spots arrayed in 48 subarrays (4000-μm2 each) and are equally spaced in vertical and horizontal directions with 16 columns and 16 rows per subarray. For the ProtoArray™ Human Protein Microarrays, spots are printed with a 250 μm spot-to-spot spacing. An extra 500 μm gap between adjacent subarrays allows quick identification of subarrays. A few of the human accessions are printed on two locations on the arrays. In such cases, results are averaged for duplicate spots at the same concentration and array location, but not across array locations.

Immune Response Biomarker Profiling Protocol

Antibodies were stored at 4° C. The tubes were barcoded upon receipt to facilitate data tracking. Microarray slides were blocked in 120 ml PBS/1% BSA/0.1% Tween 20 in glass microarray holders for 1 hour at 4° C. with gentle shaking. After blocking, arrays were removed from the blocking solution and 120 μl of each serum sample diluted 1:150 in freshly prepared Serum Profiling Buffer (1×PBS, 5 mM MgCl2, 0.5 mM DTT, 0.05% Triton X-100, 5% glycerol, 1% BSA) or buffer alone (negative control). Samples were covered with a hybrislip and allowed to incubate for 2 hours at 4C. After incubation, the hybrislip was removed and slides were inserted into Pap chambers containing 20 ml Serum Profiling Buffer. Arrays were then washed three times (8 minutes per wash) in 20 ml Profiling Buffer. Following each wash Pap chambers were inverted briefly on absorbent paper to ensure complete drainage. AlexaFluor-conjugated anti-human IgG at 1.0 mg/ml diluted in 20 ml Profiling Buffer was then added to each array and allowed to incubate with gentle tilting at 4 C for 2 hours. After incubation, the secondary antibody was removed, and arrays were washed as described above. Arrays were dried by spinning in a table top centrifuge equipped with a plate rotor at 2000 rpm for 1 minute. Arrays were then scanned using an Axon GenePix® 4000B fluorescent microarray scanner. The following negative controls were utilized in the experiment, and the data from the negative controls was pooled together for calculations related to identifying positive interacting proteins on the array: buffer=96 spots, BSA=288 spots, GST=768 spots.

Data Acquisition and Analysis

GenePix® 5.1 software (Molecular Devices Corporation) was used to overlay the mapping of human proteins in the array list file to each array image with a fixed feature size of 150 mm (diameter). After aligning each of the 48 subarrays using spots from the AlexaFluor®-conjugated and murine antibodies printed in each subarray, pixel intensities for each spot on the array were determined from the software and saved to a text file formatted for use in GenePix®, the GenePix® Result file (.gpr filename extension). These files can be opened in other text editing or spreadsheet programs.

Quantitated spot files were processed using ProtoArray™ Prospector™ software (Invitrogen Corp., Carlsbad, Calif.), incorporated by reference in its entirety, to determine which proteins interact with the serum samples. The software incorporates background correction, CI-p-Value calculations and dynamic binomial calculations of significance. CI-p-values were calculated using methods provide herein.

Results

Utilizing the calculations described above, a number of potential biomarkers were identified for melanoma. These proteins can be divided into two categories. These proteins have the potential to serve as important diagnostic or prognostic indicators (S. Brettschneider et al., Biol Psychiatry 57, 813-6 (Apr. 1, 2005)).

A number of potential biomarkers were identified in melanoma patients' serum. These proteins can be divided into two categories (Table 5). The first include proteins displaying increased reactivity in the disease population, representing the more established class of biomarkers. A total of 115 proteins were identified in this category. From this set, 101 (Table 5 in bold) meet the criteria regarding minimum frequency distributions that must be observed in order to distinguish the two populations with 95% confidence. The second category of potential biomarkers displayed decreased reactivity in the disease population relative to healthy controls.

These proteins also have the potential to serve as important diagnostic or prognostic indicators. A total of 47 proteins in this category were identified, of which 44 (Table 5 in bold and italic) meet the frequency distribution criteria described a Table 5. Numerical summary of proteins exhibiting immune reactivity in serum samples from melanoma and healthy individuals using the flexible binomial calculation of significance. Proteins highlighted in bold and bold/italic are those that can be differentiated in melanoma (M) vs. healthy (H) individuals at 95% confidence, respectively.

| H/M | | | | | | | | | | | | |
|---|---|---|---|---|---|---|---|---|---|---|---|---|
| count | 0 | 1 | 2 | 3 | 4 | 5 | 6 | 7 | 8 | 9 | 10 | Total |
| 0 | 1266 | 3 | 3 | 3 | 0 | 5 | 10 | 14 | 28 | 40 | 9 | 1381 |
| 1 | 1 | | | | | | | | | | | 1 |
| 2 | | | | | | | | | | | 1 | 1 |
| 3 | 1 | | | | | | | | | 1 | | 2 |
| 4 | | | | | | | | | 1 | | | 1 |
| 5 | 1 | | | | | | | | 0 | 2 | 1 | 4 |
| 6 | [symbol] | | | | | 1 | 1 | 3 | 0 | 9 | 1 | 23 |
| 7 | [symbol] | | | | | 0 | 2 | 1 | 2 | 8 | 1 | 21 |
| 8 | [symbol] | | | | | 1 | 1 | 1 | 4 | 19 | 5 | 39 |
| 9 | [symbol] | | | | | [symbol] | 2 | 3 | 6 | 28 | 16 | 66 |
| 10 | [symbol] | | | | 1 | 3 | 4 | 1 | 11 | 110 | 162 | 303 |
| Total | 1313 | 3 | 3 | 3 | 1 | 11 | 20 | 23 | 52 | 217 | 196 | 1842 |

While the present invention has been described in terms of the preferred embodiments, it is understood that variations and modifications will occur to those skilled in the art. Therefore, it is intended that the appended claims cover all such equivalent variations that come within the scope of the invention as claimed.

1. A method for determining a positive interaction on a protein or peptide array, comprising:
a) contacting a protein or peptide and a negative control immobilized on a protein or peptide array with a sample; and,
b) calculating a probability value for the protein or peptide using Markov's Inequality based on a comparison of a signal generated from the interaction of one or more molecules in the sample with the protein or peptide immobilized on the array and a signal generated for a negative control on the array, wherein a probability value below a threshold probability value identifies a positive interaction between the protein or peptide on the protein or peptide array and one or more molecules in the sample.

2. The method of claim 1, wherein the probability value is calculated using Chebyshev's Inequality to calculate a Chebyshev's Inequality precision value (CI-p-Value).

3. The method of claim 1, wherein the threshold probability is calculated based on experimental values obtained while performing the method.

4. The method of claim 3, wherein the threshold probability is calculated using signal values generated from negative controls on the array.

5. The method of claim 1, wherein the threshold probability is pre-set.

6. The method of claim 1, wherein the plurality of proteins or peptides are immobilized in a high density array on the solid support.

7. The method of claim 1, wherein the threshold probability is preset at a value equal to one divided by a number of protein spots on the array.

8. The method of claim 1, wherein a threshold probability value is calculated using a probability cutoff of between 0.5 and 2 false positive errors per protein or peptide array.

9. The method of claim 1, wherein the plurality of proteins or peptides are antibodies.

10. The method of claim 1, wherein the plurality of proteins or peptides comprise at least 1000 proteins from the same organism.

11. The method of claim 1, wherein the sample is a biological sample.

12. The method of claim 11, wherein the biological sample is a biological fluid.

13. The method of claim 12, wherein the biological fluid is serum or plasma.

14. A method for determining whether a binding partner is present more frequently in a first biological sample type or a second biological sample type from a plurality of biological samples of each biological sample type, comprising:
a) contacting each sample individually with a plurality of proteins or peptides immobilized on a protein array;
b) calculating a probability value for each sample for each protein or peptide on each array using Markov's Inequality based on a comparison of a signal generated from the interaction of biomolecules in each sample with each protein on each array and a signal generated for a negative control on each array;
c) identifying significant probability values using a dynamic significance calculation calculated by identifying a minimum observed probability value for each sample for each protein or peptide on each array; and
d) determining for each protein or peptide, whether a significant probability value is observed more frequently in samples of the first biological sample type or samples of the second biological sample type to identify binding partners expressed more frequently in one of the biological sample types.

15. The method of claim 14, wherein the probability value for each protein on each array is calculated using Chebyshev's Inequality to calculate a Chebyshev's Inequality precision value (CI-p-Value).

16. The method of claim 14, wherein the biological sample is a biological fluid.

17. The method of claim 16, wherein the biological fluid is serum or plasma.

18. The method of claim 14, wherein the binding partner is a polypeptide or peptide.

19. The method of claim 14, wherein the first biological sample type is a normal sample and the second biological sample type is a sample from a patient afflicted with a disease.

20. The method of claim 19 wherein the disease is an autoimmune condition, a microbiological infection, cancer, a neurological disorder, a circulatory disorder, or a respiratory disorder.

21. The method of claim 14, wherein a binding partner expressed in only one of the biological sample types are identified using a 95-99.9% confidence limit.

22. The method of claim 14, wherein a binding partner expressed in only one of the biological sample types are identified using a 97-99% confidence limit.

23. The method of claim 14, wherein the dynamic significance calculation is calculated using signal values generated from negative controls on the array.

24-28. (canceled)
